(19) United States
(12) Patent Application Publication
(10) Pub. No.: US 2011/0224638 A1
(43) Pub. Date: Sep. 15, 2011

(54) Absorbent Article With Fluorescent Wetness Indicator

(75) Inventor: Richmond R. Cohen, Williamsport, PA (US)

(73) Assignee: First Quality Products, Inc., Great Neck, NY (US)

(21) Appl. No.: 13/002,393

(22) PCT Filed: Jul. 2, 2009
(86) PCT No.: PCT/US09/49483

§ 371 Date: May 31, 2011

Related U.S. Application Data
(60) Provisional application No. 61/077,958, filed on Jul. 3, 2008.

(51) Int. Cl. A61L 15/56

(52) U.S. Cl. 604/361

(57) ABSTRACT

An absorbent article includes a body having a surface for receiving a liquid and a wetness indicator disposed on the body. The wetness indicator includes a first agent that produces a fluorescence when wetted and a second agent that produces a color change when wetted.

### FIELD OF THE INVENTION

The present invention relates to absorbent articles, and more particularly, to absorbent articles having a wetness indicator.

### BACKGROUND OF THE INVENTION

The quality of disposable absorbent article cores has advanced greatly over the years. At one time, it could be determined if the absorbent article had been wet simply by touching the outer surface of the pad, but now many absorbent articles are capable of maintaining a dry outer surface.

Absorbent articles have incorporated wetness indicators so that a caretaker can determine whether or not the absorbent article needs to be changed without disturbing the baby or incontinent patient. Wetness indicators are typically applied to the inside of the translucent backing of the absorbent article. Upon contact with urine, conventional wetness indicators provide a visual signal to indicate the presence of the urine, such as by changing color. The visual signal may be triggered by the presence of moisture or by a pH shift to the colorant.

Although conventional wetness indicators provide a means for a caretaker to detect wetness in the absorbent article during the day time, the wetness indicators are not effective in darkness at night. It is a common practice in many long-term care facilities for caregivers to turn on the lights every two hours at night to check whether an incontinent resident's absorbent article needs to be changed. These constant checks may awaken the resident, resulting in sleep deprivation, which is one of the most detrimental conditions afflicting long-term care residents today. Analogously, a parent may have to disturb an infant sleeping in darkness to check on the wetness of an absorbent article.

Thus, there is a need for an absorbent article containing a wetness indicator which is easily detectable either in the day or at night, and which is both convenient and practical to produce.

### SUMMARY OF THE INVENTION

According to one embodiment, an absorbent article is provided including a body having a surface for receiving a liquid and a wetness indicator disposed on the body. The wetness indicator includes a first agent that produces a fluorescence when wetted and a second agent that produces a color change when wetted.

In at least one embodiment, the fluorescence is perceptible when exposed to ultraviolet light.

In at least one embodiment, the color change is perceptible under visible light.

In at least one embodiment, the wetness indicator is arranged in a predetermined pattern when dry, and disperses into an irregular pattern when wetted.

In at least one embodiment, the wetness indicator comprises a hot-melt adhesive.

In at least one embodiment, the body includes a liquid-permeable topsheet, a liquid-impermeable backsheet, and an absorbent core disposed between the topsheet and the backsheet.

In at least one embodiment, the wetness indicator is disposed between the absorbent core and the interior facing side of the backsheet.

In at least one embodiment, at least a portion of the backsheet is translucent.

In at least one embodiment, the backsheet comprises a film. The backsheet may further comprise a nonwoven material attached to the film.

In at least one embodiment, the absorbent article is a diaper. The diaper may be an adult diaper.

In at least one embodiment, the first agent comprises at least one of an acridine dye, cyanine dye, xanthene dye, pyrene dye, benzoxazole dye, fluoranthene dye, and quinine dye.

In at least one embodiment, the first agent comprises at least one of Fluorescein salt, and Pyranine 10G.

According to one embodiment, a method of manufacturing an absorbent article includes forming a body having a surface for receiving a liquid. A wetness indicator is disposed on the body. The wetness indicator includes a first agent that produces a fluorescence when wetted, and a second agent that produces a color change when wetted.

In at least one embodiment, forming the body includes forming a liquid-permeable topsheet and forming a liquid-impermeable backsheet. An absorbent core is disposed between the topsheet and the backsheet.

In at least one embodiment, the wetness indicator is disposed between the absorbent core and an interior facing side of the backsheet.

In at least one embodiment, the wetness indicator comprises a hot-melt adhesive.

In at least one embodiment, at least a portion of the backsheet is translucent.

In at least one embodiment, the backsheet comprises a film. The backsheet may further comprise a nonwoven material.

In at least one embodiment, the fluorescence is perceptible when exposed to ultraviolet light.

In at least one embodiment, the color change is perceptible under visible light.

In at least one embodiment, the wetness indicator is disposed in a predetermined pattern and is dispersible into an irregular pattern when wetted.

In at least one embodiment, the first agent comprises at least one of an acridine dye, cyanine dye, xanthene dye, pyrene dye, benzoxazole dye, fluoranthene dye, and quinine dye.

In at least one embodiment, the first agent comprises at least one of Fluorescein salt, and Pyranine 10G.

These and other features of this invention are described in, or are apparent from, the following detailed description of various exemplary embodiments of this invention.

### BRIEF DESCRIPTION OF THE DRAWINGS

Exemplary embodiments of this invention will be described with reference to the accompanying figures.

FIG. 1 is a perspective view of an absorbent article according to an exemplary embodiment of the present invention.

FIG. 2 is a cross-section view of the absorbent article of FIG. 1.

### DETAILED DESCRIPTION OF THE EXEMPLARY EMBODIMENTS

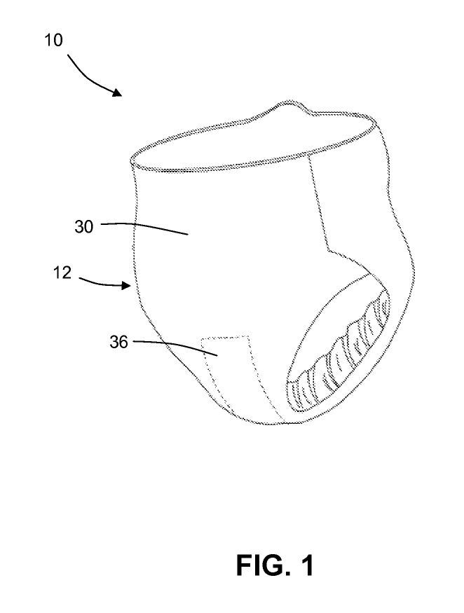

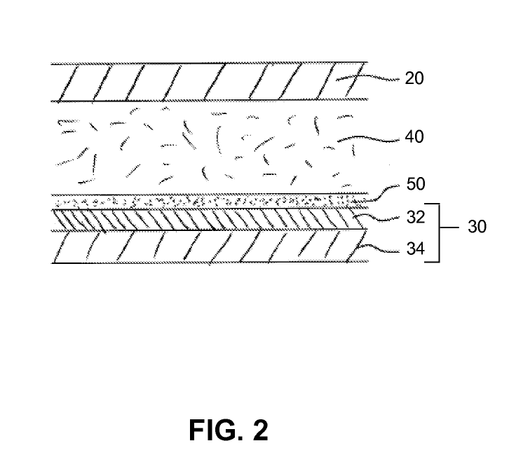

FIG. 1 is a perspective view of an absorbent article according to an exemplary embodiment of the present invention. FIG. 2 is a cross-section view of the absorbent article of FIG. 1.

An absorbent article 10 comprises a body 12 including a liquid-permeable nonwoven topsheet 20, a liquid permeable backsheet 30, and an absorbent core 40 interposed between the topsheet 20 and the backsheet 30. A wetness indicator 50 is disposed between the backsheet 30 and the absorbent core 40.

The backsheet 30 is comprised of a film 32. Preferably, the film 32 is a polyolefin film, and more preferably, a polyethylene film. The film 32 may be laminated to a nonwoven material 34. At least a portion 36 of the backsheet 30 is translucent so that a visual signal from the wetness indicator 50 can be seen through the backsheet 30 from the underside of the absorbent article 10.

The wetness indicator 50 may be a wetness indicating adhesive. Preferably, the wetness indicator 50 is a hot-melt wetness indicating adhesive. For example, the hot-melt wetness indicating adhesive may be, for example, product number H9486F, manufactured by Bostik, Inc. of Wauwatosa, Wis.

The wetness indicator 50 is applied by a conventional method known in the art, such as extruding, slotting, spraying, printing, or the like. For example, the wetness indicator 50 may be applied on the translucent portion of the film 32 on the inside facing side of the backsheet 30. The wetness indicator 50 may contact both the absorbent core 40 and the film portion of the backsheet 30, thus when the wetness indicator 50 is an adhesive, it will act to secure together the absorbent core 40 and the backsheet 30.

The wetness indicator 50 need not cover a large portion of the interior facing side of the backsheet 30. For example, the wetness indicator 50 may be applied in one or more slotted lines along the length of the backsheet. The slotted lines may be optionally intermittent.

The wetness indicator 50 includes a fluorescing wetness indicating agent. The fluorescing wetness indicating agent is used in an amount effective to provide the composition with a fluorescence which can easily be seen in the dark to indicate that the absorbent article has been wetted. The fluorescing wetness indicating agent may fluoresce only when wet, and not when dry. By the term “wet” or “wetted” it is meant that the adhesive comes into contact with a water-based fluid, such as urine, saline solution, blood, mucous, and other bodily exudates, as well as water itself.

The fluorescing wetness indicating agent is preferably water soluble such that it is activated to produce a fluorescence in response to contact with a liquid. For example, the fluorescing agent is inactive when the wetness indicator is dry. When the wetness indicator is contacted with or dissolved in a liquid, the fluorescing agent is activated such that it produces a fluorescence. The fluorescing wetness indicating agent is preferably contained in the hot-melt adhesive so that it is only activated upon exposure to an aqueous environment.

The fluorescing wetness indicating agent may produce a fluorescence that is visible to the human eye in the dark and/or under light, such as visible light, infrared light, or ultraviolet light. However, it is preferred that the fluorescing agent is an ultraviolet (UV) fluorescent wetness indicating agent which produces a fluorescence that is visible to the human eye when irradiated by UV light from a UV light source (e.g., a black light).

Suitable fluorescing wetness indicating agents include, for example, known compounds having fluorescent properties such as those disclosed in U.S. Pat. Nos. 3,941,759 to Taylor et al., 4,841,156 to May et al., 5,667,840 to Tingey et al., 6,080,450 to Canor, 6,391,281 to Rueggeberg et al., and 6,461,326 to Yang et al., which are hereby incorporated by reference. For example, the fluorescing wetness indicating agent may comprise an acridine dye, such as Acridine Orange and Acridine Yellow; cyanine dye, such as Cy3 and Cy5; xanthene dye, such as Eosin, Fluorescein, and Rhodamine; pyrene dye, such as Alex Fluor® and AMCA-X; benzoxazole dye, such as Uvitex® OB; fluoranthene dye; quinine dye; and the like. Preferably, the fluorescing agent is a water soluble fluorescing agent, such as Fluorescein, Fluorescein sodium salt, and Pyranine 10G.

The wetness indicator 50 further includes a conventional wetness indicating agent that produces a visual signal perceptible to the human eye under visible light to indicate that the absorbent article has been wetted. For example, the visual signal may be a change in color, such as from a first color to a second color, from one shade of a color to a different shade of the color, from a lighter or translucent color to a darker color, etc. Useful wetness indicating agents include dyestuffs or colorants and pH indicators which are capable of changing the color of the adhesive composition when wetted. The conventional wetness indicating agent is used in an amount effective to provide the composition with a readily visible color when wet that is distinguishable from the color of the dry composition.

It is preferred that the conventional wetness indicating agent is an acid-base wetness indicator that changes color in response to changes in pH when contacted by a liquid, such as urine. Suitable conventional acid-base wetness indicating agents include, for example, known wetness indicating agents such as those described in U.S. Pat. Nos. 5,066,711 to Colon et al. and 6,904,865 to Klofta et al., which are hereby incorporated by reference. For example, the conventional acid-base wetness indicating agent may comprise a sulfonepthalein pH indicator, such as Ethyl Red, Bromophenol Blue, Bromochlorophenol Blue, Bromocresol Green, Bromocresol, M-cresol Purple, Cresol Red, Chlorophenol Red, Bromothymol Blue, Bromopyrogallol red, and Bromoxylenol Blue; monoazo dye, such as acid alizarin violet N; monoazo pyrazoline dye, such as acid yellow 34; diazo dye, such as acid black 24; amphoteric anthraquinone dye, such as acid black 48; amphoteric anthraquinone dye, such as acid blue 45; triphenylmethane dye, such as acid fuchsin; phthalein type dye, such as o-cresolphtalein; xanthene dye, such as 2′,7′-dicholorofluorescein eosin B; heterocylic acridine aromatics, such asacridine orange; diphenyl methane dye, such as auromine O; triphenylmethane dye, such as basic fuchsin; cationic thiazine dye, such as azure C; cationic anthraquinone dye, such as basic blue 47; phthalocyanine type dye, such as copper phthalocyanine; quaternized phthalocyanine type dye, such as alcec blue; cationic polymethine dye, such as astrazon orange G; anthraquinone type dye, such as alizarin; neutral complex dye, such as azure A eosinate; terpene type dyes, such as trans-beta-carotene; and the like. The most preferred acid-base wetness indicators include Bromophenal Blue, Bromochlorophenol Blue, Bromocresol Green, and Bromocresol purple.

The conventional wetness indicating agent may also be an indicator that changes color when contacted by a liquid. Suitable conventional wetness indicating agents that change color when contacted by or dissolved in a liquid include, for example, known wetness indicating agents such as those described in U.S. Pat. Nos. 3,675,654 to Baker et al. and 5,342,861 to Raykovitz, which are hereby incorporated by reference. For example, the conventional wetness indicating agent may comprise a water soluble dye, such as a nitro dye, monoazo dye, diazo dye, phthalocyanine dye, quinoline dye, xanthene dye, triaryl methane dye, indigoid dye, vegetable dye, food dye, and the like.

The wetness indicator 50 is preferably water soluble or partially water soluble. For example, the wetness indicator 50 may comprise a water soluble polymer, such as a high molecular weight polyethylene glycol.

In one embodiment, the water solubility of the composition of the wetness indicator 50 provides for selective control of the fluorescence of the wetness indicator 50. For example, when the wetness indicator 50 is dry, the water soluble matrix of the wetness indicator 50 contains the fluorescing agent to prevent the fluorescing agent from fluorescing. When the wetness indicator 50 is insulted with a liquid, the water soluble matrix releases the fluorescing agent to contact the liquid, thereby allowing it to fluoresce. The water solubility of the composition of the wetness indicator 50 may also be used to provide for selective control of the color change of the conventional wetness indicating agent.

In another embodiment, the water solubility of the composition of the wetness indicator 50 allows the wetness indicator 50 to change configurations to indicate when wetted. For example, when the wetness indicator 50 is dry, the wetness indicator 50 is fixed in place in a predetermined pattern according to how the wetness indicator 50 was applied, e.g. a straight line. When the wetness indicator 50 is insulted with a liquid, the water soluble matrix is dispersed into an irregular pattern.

Other compatible components that have a desired effect on the properties of the wetness indicator 50 may be included therein. For example, the wetness indicator 50 may include a tackifier, such as rosin based tackifying resin; a surfactant, such as a fatty acid ester; a plasticizer, such as liquid benzoate plasticizer; a stabilizer or antioxidant, such as hindered phenolic antioxidant; a wax, and the like.

An absorbent article having a fluorescing wetness indicator provides a mechanism for visually indicating when the absorbent article has been insulted in the dark and/or when exposed to UV light. Further, an absorbent article having a wetness indicator including a fluorescing wetness indicating agent and a conventional wetness indicating agent provides a dual-signal mechanism for visually indicating when the absorbent article has been insulted under different lighting conditions. The fluorescing wetness indicating agent provides a visual signal perceptible in the dark and/or when exposed to UV light, while the conventional wetness indicating agent provides a visual signal perceptible under visible light.

The present invention may be applied to any number of absorbent articles, including, but not limited to, baby diapers, training pants, children's diapers, adult diapers, adult briefs, adult protective underwear, overnight protective underwear for children, underpads, and the like. It will be understood that the present invention may be applied to other types of applications differing from the type described above.

Now that exemplary embodiments of the present invention have been shown and described in detail, various modifications and improvements thereon will become readily apparent to those skilled in the art. Accordingly, the spirit and scope of the present invention is to be construed broadly and limited only by the appended claims, and not by the foregoing specification.

What is claimed is:

1. An absorbent article comprising:
a body having a surface for receiving a liquid; and
a wetness indicator disposed on said body, said wetness indicator comprising:
a first agent producing a fluorescence when wetted, and
a second agent producing a color change when wetted.

2. The absorbent article of claim 1, wherein said fluorescence is perceptible when exposed to ultraviolet light.

3. The absorbent article of claim 1, wherein said color change is perceptible under visible light.

4. The absorbent article of claim 1, wherein said wetness indicator is arranged in a predetermined pattern when dry, and dispersible into an irregular pattern when wetted.

5. The absorbent article of claim 1, wherein said wetness indicator comprises a hot-melt adhesive.

6. The absorbent article of claim 1, wherein said body comprises:
a liquid-permeable topsheet;
a liquid-impermeable backsheet; and
an absorbent core disposed between said topsheet and said backsheet.

7. The absorbent article of claim 6, wherein said wetness indicator is disposed between said absorbent core and an interior facing side of said backsheet.

8. The absorbent article of claim 6, wherein at least a portion of said backsheet is translucent.

9. The absorbent article of claim 6, wherein said backsheet comprises a film.

10. The absorbent article of claim 9, wherein said backsheet further comprises a nonwoven material.

11. The absorbent article of claim 1, wherein said absorbent article comprises a diaper.

12. The absorbent article of claim 11, wherein said diaper is an adult diaper.

13. The absorbent article of claim 1, wherein said first agent comprises at least one of an acridine dye, cyanine dye, xanthene dye, pyrene dye, benzoxazole dye, fluoranthene dye, and quinine dye.

14. The absorbent article of claim 1, wherein said first agent comprises at least one of Fluorescein, Fluorescein salt, and Pyranine 10G.

15. A method of manufacturing an absorbent article, comprising:
forming a body having a surface for receiving a liquid; and
disposing a wetness indicator on said body, said wetness indicator comprising:
a first agent producing a fluorescence when wetted, and
a second agent producing a color change when wetted.

16. The method of claim 15, wherein forming said body comprises:
forming a liquid-permeable topsheet;
forming a liquid-impermeable backsheet; and
disposing an absorbent core between said topsheet and said backsheet.

17. The method of claim 16, wherein said wetness indicator is disposed between said absorbent core and an interior facing side of said backsheet.

18. The method of claim 16, wherein said wetness indicator comprises a hot-melt adhesive.

19. The method of claim 16, wherein at least a portion of said backsheet is translucent.

20. The method of claim 16, wherein said backsheet comprises a film.

21. The method of claim 20, wherein said backsheet further comprises a nonwoven material.

22. The method of claim 15, wherein said fluorescence is perceptible when exposed to ultraviolet light.

23. The method of claim 15, wherein said color change is perceptible under visible light.

24. The method of claim 15, wherein said wetness indicator is disposed in a predetermined pattern, and is dispersible into an irregular pattern when wetted.

25. The method of claim 15, wherein said first agent comprises at least one of an acridine dye, cyanine dye, xanthene dye, pyrene dye, benzoxazole dye, fluoranthene dye, and quinine dye.

26. The method of claim 15, wherein said first agent comprises at least one of Fluorescein, Fluorescein salt, and Pyranine 10G.
